(19) United States
(12) Patent Application Publication
(10) Pub. No.: US 2003/0236533 A1
(43) Pub. Date: Dec. 25, 2003

(54) Shape memory polymer actuator and catheter

(75) Inventors: Thomas S. Wilson, Castro Valley, CA (US); Duncan Maitland, Pleasant Hill, CA (US); Daniel L. Schumann, Concord, CA (US); Steve L. Little, Tracy, CA (US); Paul E. Ahre, Livermore, CA (US)

Correspondence Address: Eddie E. Scott, Assistant Laboratory Counsel, Lawrence Livermore National Laboratory, P.O. Box 808, L-703, Livermore, CA, 94551, US

(73) Assignee: The Regents of the University of California

(21) Appl. No.: 10/177,225

(22) Filed: Jun. 20, 2002

(51) Int. Cl. A61B 17/22

(52) U.S. Cl. 606/127; 606/200

(57) ABSTRACT

A system for removing an occlusion from a vessel. The system utilizes a shape memory polymer device for acting upon the occlusion. A transport vehicle moves a shape memory material through the vessel. The shape memory material is adapted to move from a first unexpanded coil shape to move through or around the occlusion, to a second expanded coil shape for acting upon the occlusion. A heat transfer mechanism is operatively connected to the shape memory material and is adapted to transfer heat to the shape memory material to move the shape memory material from the first shape to the second shape. The transport vehicle and the shape memory polymer material is withdrawn through the conduit carrying the occlusion matter. The system may include a second shape memory polymer device situated so that the occlusion material is gripped from both sides to facilitate removal.

### CROSS-REFERENCE TO RELATED APPLICATIONS

Related subject matter is disclosed and claimed in the following commonly owned, copending, U.S. patent applications; U.S. patent application Ser. No. 09/761024, by Duncan J. Maitland filed Jan. 16, 2001 titled “Light Diffusing Fiber Optic Chamber” and U.S. patent application Ser. No. 09/761023, by Duncan J. Maitland, Abraham P. Lee, Daniel L. Schumann, Dennis Matthews, Derek Decker, and Charles Jungreis, filed Jan. 16, 2001 titled “Shape Memory Polymer Actuator and Catheter.” The commonly owned, copending, U.S. patent applications identified above are incorporated herein by reference in their entirety.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to catheters and in particular to a catheter with a shape memory polymer actuator.

2. State of Technology

U.S. Pat. No. 5,836,868 for an expandable intravascular occlusion material removal devices and methods of use, by Ressemann, et al., patented Nov. 17, 1998, provides the following description: “The present invention generally relates to constructions for intravascular treatment devices useful for removing vascular occlusion material from a vascular occlusion or from a vascular lumen. The invention more specifically relates to expandable intravascular occlusion material removal devices, as well as to methods of using those devices to treat vascular diseases.

Vascular diseases, such as atherosclerosis and the like, have become quite prevalent in the modem day. These diseases may present themselves in a number of forms. Each form of vascular disease may require a different method of treatment to reduce or cure the harmful effects of the disease. Vascular diseases, for example, may take the form of deposits or growths in a patient's vasculature which may restrict, in the case of a partial occlusion, or stop, in the case of a total occlusion, blood flow to a certain portion of the patient's body. This can be particularly serious if, for example, such an occlusion occurs in a portion of the vasculature that supplies vital organs with blood or other necessary fluids.

To treat these diseases, a number of different therapies are being developed. While a number of invasive therapies are available, it is desirable to develop non-invasive therapies as well. Non-invasive therapies may be less risky than invasive ones, and may be more welcomed by the patient because of the possibility of decreased chances of infection, reduced post-operative pain, and less post-operative rehabilitation. One type of non-invasive therapy for vascular diseases is pharmaceutical in nature. Clot-busting drugs have been employed to help break up blood clots which may be blocking a particular vascular lumen. Other drug therapies are also available. Further non-invasive, intravascular treatments exist that are not only pharmaceutical, but also revascularize blood vessels or lumens by mechanical means. Two examples of such intravascular therapies are balloon angioplasty and atherectomy which physically revascularize a portion of a patient's vasculature.

Balloon angioplasty comprises a procedure wherein a balloon catheter is inserted intravascularly into a patient through a relatively small puncture, which may be located proximate the groin, and intravascularly navigated by a treating physician to the occluded vascular site. The balloon catheter includes a balloon or dilating member which is placed adjacent the vascular occlusion and then is inflated. Intravascular inflation of the dilating member by sufficient pressures, on the order of 5 to 12 atmospheres or so, causes the balloon to displace the occluding matter to revascularize the occluded lumen and thereby restore substantially normal blood flow through the revascularized portion of the vasculature. It is to be noted, however, that this procedure does not remove the occluding matter from the patient's vasculature, but displaces it.

While balloon angioplasty is quite successful in substantially revascularizing many vascular lumens by reforming the occluding material, other occlusions may be difficult to treat with angioplasty. Specifically, some intravascular occlusions may be composed of an irregular, loose or heavily calcified material which may extend relatively far along a vessel or may extend adjacent a side branching vessel, and thus are not prone or susceptible to angioplastic treatment. Even if angioplasty is successful, thereby revascularizing the vessel and substantially restoring normal blood flow therethrough, there is a chance that the occlusion may recur. Recurrence of an occlusion may require repeated or alternative treatments given at the same intravascular site.

Accordingly, attempts have been made to develop other alternative mechanical methods of non-invasive, intravascular treatment in an effort to provide another way of revascularizing an occluded vessel and of restoring blood flow through the relevant vasculature. These alternative treatments may have particular utility with certain vascular occlusions, or may provide added benefits to a patient when combined with balloon angioplasty and/or drug therapies.

One such alternative mechanical treatment method involves removal, not displacement, as is the case with balloon angioplasty, of the material occluding a vascular lumen. Such treatment devices, sometimes referred to as atherectomy devices, use a variety of means, such as lasers, and rotating cutters or ablaters, for example, to remove the occluding material. The rotating cutters may be particularly useful in removing certain vascular occlusions. Since vascular occlusions may have different compositions and morphology or shape, a given removal or cutting element may not be suitable for removal of a certain occlusion. Alternatively, if a patient has multiple occlusions in his vasculature, a given removal element may be suitable for removing only one of the occlusions. Suitability of a particular cutting element may be determined by, for example, its size or shape. Thus, a treating physician may have to use a plurality of different treatment devices to provide the patient with complete treatment. This type of procedure can be quite expensive because multiple pieces of equipment may need to be used (such intravascular devices are not reusable because they are inserted directly into the blood stream), and may be tedious to perform because multiple pieces of equipment must be navigated through an often-tortuous vascular path to the treatment site.”

U.S. Pat. No. 5,102,415, for an apparatus for removing blood clots from arteries and veins, by Guenther, et al., patented Apr. 7, 1992, provides the following description: “A triple catheter for removing of blood clots from arteries and veins is equipped with an outer catheter that can be inserted into a blood vessel and an inner catheter with an inflatable balloon at its distal end that can be inserted into the outer catheter. The inner catheter is surrounded by an intermediate catheter also inserted into the outer catheter. The intermediate catheter has a radially expandable distal end receptacle made of an elastic mesh structure of spring wires or plastic monofilaments covered by or embedded in an elastic plastic coating. A very small puncture channel is required for the insertion of such a triple catheter through the wall of a blood vessel.”

U.S. Pat. No. 5,645,564 for microfabricated therapeutic actuator mechanisms, by Northrup, et al., patented Jul. 8, 1997, provides the following description: “Electromechanical microstructures (microgrippers), either integrated circuit (IC) silicon-based or precision machined, to extend and improve the application of catheter-based interventional therapies for the repair of aneurysms in the brain or other interventional clinical therapies. These micromechanisms can be specifically applied to release platinum coils or other materials into bulging portions of the blood vessels also known as aneurysms. The “micro” size of the release mechanism is necessary since the brain vessels are the smallest in the body. Through a catheter more than one meter long, the micromechanism located at one end of the catheter can be manipulated from the other end thereof. The microgripper (micromechanism) of the invention will also find applications in non-medical areas where a remotely actuated microgripper or similar actuator would be useful or where micro-assembling is needed.”

U.S. Pat. No. 6,102,917 for a shape memory polymer (SMP) gripper with a release sensing system, by Maitland, et al., patented Aug. 15, 2000, provides the following description: “A system for releasing a target material, such as an embolic coil from an SMP located at the end of a catheter utilizing an optical arrangement for releasing the material. The system includes a laser, laser driver, display panel, photodetector, fiber optics coupler, fiber optics and connectors, a catheter, and an SMP-based gripper, and includes a release sensing and feedback arrangement. The SMP-based gripper is heated via laser light through an optic fiber causing the gripper to release a target material (e.g., embolic coil for therapeutic treatment of aneurysms). Various embodiments are provided for coupling the laser light into the SMP, which includes specific positioning of the coils, removal of the fiber cladding adjacent the coil, a metal coating on the SMP, doping the SMP with a gradient absorbing dye, tapering the fiber optic end, coating the SMP with low refractive index material, and locating an insert between the fiber optic and the coil.”

U.S. Pat. No. 5,843,118 for fibered micro vaso-occlusive devices, by Sepetka, et al., patented Dec. 1, 1998, provides the following description: “This is a vaso-occlusive device made up of at least one short retainer and a longer fiber bundle. The retainer may be radio-opaque. The fibers may be straight, looped, or tufted. The primary use of the device is in the very small vessels at the distal portion of the vasculature.”

U.S. Pat. No. 5,895,398 for a method of using a clot capture coil, by Wensel, et al., patented Apr. 20, 1999, provides the following description: “A clot and foreign body removal device is described which comprises a catheter with at least one lumen. Located within the catheter is a clot capture coil that is connected to an insertion mandrel. In one embodiment, the clot capture coil is made out of a solid elastic or superelastic material which has shape memory, preferably nitinol. The elasticity or superelasticity of the coil allows it to be deformed within the catheter and to then reform its original coil configuration when the coil is moved outside of the catheter lumen. In another embodiment the coil is a biphasic coil which changes shape upon heating or passing an electric current. Once the coil configuration has been established, the coil can be used to ensnare and corkscrew a clot in a vessel. A clot is extracted from the vessel by moving the clot capture coil and catheter proximally until the clot can be removed or released into a different vessel that does not perfuse a critical organ. Foreign bodies are similarly captured by deploying the coil distal to the foreign body and moving the clot capture coil proximally until the foreign body is trapped within the coil. By removing the device from the body, the foreign material is also removed.”

Patents that provide additional background information include: U.S. Pat. No. 3,868,956 to Alfidi, Ralph J. and Cross, William B., “Vessel implantable appliance and method of implanting it,” Mar. 4, 1975; U.S. Pat. No. 3,996,938 to Clark, W. T., “Expanding mesh catheter,” Dec. 14, 1976; U.S. Pat. No. 4,140,126 to Choudhury, M. H., “Method for performing aneurysm repair,” Feb. 20, 1979; U.S. Pat. No. 4,706,671 to Weinrib, H. P., “Catheter with coiled tip,” Nov. 17, 1987; U.S. Pat. No. 4,873,978 to Ginsburg, R., “Device and method for emboli retrieval,” Oct. 17, 1989; U.S. Pat. No. 5,011,488 to Ginsburg, R., “Thrombus extraction system,” Apr. 30, 1991; U.S. Pat. No. 5,049,591 to Hayashi, S. and Fujimori, H. “Shape Memory Polymer Foam,” Sep. 17, 1991; U.S. Pat. No. 5,102,415 to Guenther, R. W. and Vorwerk, D., “Apparatus for removing blood clots from arteries and veins,” Apr. 7, 1992; U.S. Pat. No. 5,330,483 to Heaven, M. D., and Schuler, M., “Specimen reduction device,” Jul. 9, 1994; U.S. Pat. No. 5,370,609 to Drasler, W. J., Dutcher, R. G., Jenson, M. L., Thielen, J. M., Protonotarios, E. I., “Thrombectomy device,” Dec. 6, 1994; U.S. Pat. No. 5,411,509 to Hilal, S., “Embolectomy catheter,” May 2, 1995; U.S. Pat. No. 5,490,859 to Mische, H. A., Ressemann, T. V., Vrba, A. C., and Hackett, S. S., “Expandable intravascular occlusion materials removal devices and methods of use,” Feb. 13, 1996; U.S. Pat. No. 5,603,722 to Phan, L., Froix, M. and Stertzer, S., “Intravascular stent,” Feb. 18, 1997; U.S. Pat. No. 5,674,242 to Phan, L., et al., “Endoprosthetic Device With Therapeutic Compound,” Oct. 7, 1997; U.S. Pat. No. 5,762,630 to Bley, R., and Kubacki, G., “Thermally softening stylet,” Jun. 9, 1998; U.S. Pat. No. 5,792,157 to Mische, H. A., Ressemann, T. V., Hoium, S. A., “Expandable intravascular occlusion material removal devices and methods of use,” Aug. 11, 1998; U.S. Pat. No. 5,846,247 to Unsworth, J. D., and Waram, T. C., “Shape memory tubular deployment system,” Dec. 8, 1998; U.S. Pat. No. 5,897,567 to Ressemann, T. V., Vrba, A. C., Hackett, S. S., Kugler, C. J., Mische, H. A., “Expandable intravascular occlusion material removal devices and methods of use,” Apr. 27, 1999; U.S. Pat. No. 5,902,518 to Khazai, B. and Nichols, G. M., “Self-regulating polymer composite heater,” May 11, 1999; U.S. Pat. No. 5,910,357 to Hachisuka, H., Kondo, Y., Ikeda, K., Takano, H., Mochisuki, H., “Separation membrane and method of producing the same, and shape memory composition,” Jun. 8, 1999; U.S. Pat. No. 5,957,966 to Schroeppel, E. A., Spehr, P. R., and Machek, J. E., “Implantable cardiac lead with multiple shape memory polymer structures,” Sep. 28, 1999; U.S. Pat. No. 5,964,744 to Balbierz, D. J. Walker, J. M., Thomas, J. R., Bley, R. S., Van Bladel, K., “Polymeric medical devices having shape memory,” Oct. 12, 1999; U.S. Pat. No. 6,022,309 to Celliers, P., Da Silva, L., Glinsky, M., London, R., Maitland, D., Matthews, D., Fitch, P., “Optoacoustic thrombolysis,” Feb. 8, 2000; U.S. Pat. No. 6,086,599 to Lee, A. P., Fitch, “Micro devices using shape memory polymer patches for mated connections,” Jul. 11, 2000; U.S. Pat. No. 6,090,072 to Kratoska, W. F., Tay, S.-W., Thome, S. P., Keith, P. T., “Expandable introducer shealth.” Jul. 18, 2000; U.S. Pat. No. 6,102,933 to Lee, A. P., Northrup, A., Ciarlo, D. R., Krulevitch, P. A., and Bennett, W. J., “Release mechanism utilizing shape memory polymer material,” Aug. 15, 2000; U.S. Pat. No. 6,120,515 to Rogers, L., Buckley, J. T., Hundermark, R. R., Powell, F. T., Milo, C., and Castro, A. J., “Composite Atherectomy Cutter,” Sep. 19, 2000; EP0337918B1 to Monfort, M. Y, Molenauer, K. M., and Chin, A. K., “Endarectomy apparatus,” Nov. 9, 1994; EP0472368B1: Fearnot, N. E., “Ablation catheter,” Jun. 28, 1995; and W00003643: Maitland, D. J., Lee, A. P., Schumann, D. L., and Da Silva, L., “Shape memory polymer gripper with a release sensing system,” Jan. 27, 2000.

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for removing matter from a conduit. The system includes the steps of passing a transport vehicle and a shape memory polymer material through the conduit, passing the shape memory polymer material through or around the matter, transmitting energy to the shape memory polymer material for moving the shape memory polymer material from a first shape to a second and different shape, and withdrawing the transport vehicle and the shape memory polymer material through the conduit carrying the matter. The system utilizes a shape memory polymer device for acting upon a material in a conduit. A transport vehicle is adapted to move through the conduit. A shape memory material is operatively connected to the transport vehicle. The shape memory material is adapted to move from a first shape to move through or around the material, to a second and different shape for acting upon the material. A heat transfer mechanism is operatively connected to the shape memory material and is adapted to transfer heat to the shape memory material to move the shape memory material from the first shape to the second shape. The transport vehicle and the shape memory polymer material is withdrawn through the conduit carrying the matter.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIGS. 1 and 2 illustrates an embodiment of the present invention that provides an expandable device situated at the distal end of a catheter used in a system for the removal of vascular and non-vascular occlusions.

FIGS. 3A and 3B illustrate a system that provides another embodiment of an expandable device situated at the distal end of a catheter used in a system for the removal of vascular and non-vascular occlusions.

FIG. 4 illustrates additional details of an expandable device situated at the distal end of a catheter used in a system for the removal of vascular and non-vascular occlusions.

FIG. 5 is a schematic overview of an ischemic stroke treatment system.

### DETAILED DESCRIPTION OF THE INVENTION

Referring now to the drawings, to the following detailed information, and to incorporated materials; a detailed description of the invention, including specific embodiments, is presented. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The present invention provides an expandable device that can be used to help grip objects. Embodiments of the present invention provide an expandable device situated at the distal end of a catheter used in a system for the removal of vascular and non-vascular occlusions. Various embodiments of systems that provide an expandable device used in a the removal of vascular and non-vascular occlusions are illustrated in the drawings. The expandable device is constructed of a shape memory polymer (SMP). A SMP is a polymer which can be formed into a primary or equilibrium shape, re-formed into a stable secondary or stressed shape, and actuated by various means to recover its primary shape.

Thromboembolic disorders, such as stroke, pulmonary embolism, thrombosis, and arteriosclerosis are a major cause of morbidity and mortality in the United States. Such disorders are characterized by the partial or complete occlusion of a blood vessel by clots, fatty deposits, or plaque. The tissue downstream of the occluded artery becomes ischemic (oxygen and nutrient deprived). If the occlusion persists, the condition will gradually worsen with time until the cell death (infarction) occurs. The end result, if circulation is not restored, can be loss of limb, angina pectoris, myocardial infarction, stroke, or death.

There are several existing techniques to re-establish flow in occluded vascular passageways and these include balloon angioplasty, balloon embolectomy, and catheter based percutaneous methods such as treatment with thrombolytic drugs, various types of atherectomy, and removal of thromboembolic material with capture coils and like devices.

In balloon angioplasty, a balloon tipped catheter is non-surgically introduced into the vasculature and advanced to the point of occlusion. The balloon section of the catheter is advanced into the stenotic region and next inflated to dilate the blockage. Balloon angioplasty then does not remove the material causing the occlusion, which are subject to future complications such as restenosis or embolic events and is especially unsuitable for smaller vessels.

A common technique for treatment of clots is to place a microcatheter near the clot and infuse a thrombolytic material such as streptokinase, urokinase, or recombinant tissue plasminogen activator (rTPA) to dissolve the clot. However, thrombolysis typically takes hours to days to remove the clot and is typically ineffective unless administered within approximately 3 hours of the thromboembolic event. Thrombolytics also can cause severe hemoraging in patients, especially if used after the initial three hour use window, and cannot be used at all in some patients.

One of the first percutaneous methods for recanalization through vascular occlusions involved balloon embolectomy. A catheter with a balloon tip on the distal end is advanced to the site of occlusion through a guide catheter. The distal end is advanced past the site of the occlusion and the balloon is inflated to fill the vascular passageway. It is then pulled back toward the guide catheter, dislodging embolic material from the vascular lumen while doing so. The occluding material is then withdrawn into the guide catheter by the pushing action of the balloon. While devices for balloon embolectomy have improved over the years, there remain problems such as being able to effectively separate material from the vascular lumen and removing the occlusion without it breaking into fragments which can move downstream and lodge in a smaller vessel, causing another thromboembolic event. There are also size limitations on using balloons for expansion of the distal end, preventing this type of device from being usable in small vessels.

Various percutaneous techniques for atherectomy have been developed based on mechanical, hydrodynamic, and acoustic methods for breaking up the occlusion and removal of the resulting debris. For example, several devices have been developed using a high speed rotary abrasion tool comprising abrasive embedded wires or wire meshes which can be controllably expanded by balloon action or longitudinal compression of the wire mesh resulting in circumferential expansion. These devices mechanically grind the occluding material into small fragments which can be withdrawn back up through the catheter via suction. However, such devices are limited to larger size vessels due to limits in construction, being able to move the devices to a smaller tortuous lumen, and in being able to transmit torque once the device falls below a certain size.

Another atherectomy device is described in U.S. Pat. No. 5,370,609 which uses a high velocity jet of saline to create high local shear stresses at the catheter tip, in turn breaking the clot into small pieces. This device has advantages of not having any moving parts, of high speed, flexible for moderate vessels, and good removal of clot debris. However, current designs are unable to be used in vessels smaller than 3 mm, while most embolic strokes occur in vessels smaller than this.

Acoustic wave based thrombectomy devices have also been developed for recanalization of blocked vessels. Acoustic wave generating devices are used at the distal end of the catheter to break the occlusive material into very small pieces, which can then be either suctioned out or in some cases left for re-absoption by the body. Such devices are still experimental and not yet being used commercially.

Embolic coil and other clot capture devices have been developed for thrombectomy as shown by U.S. Pat. Nos. 4,706,671; 4,873,978 and 5,895,398. These devices operate in a manner similar to the balloon embolectomy, but using means for retrieving the clot.

Shape-memory materials have the useful ability of being formable into a primary shape, being reformable into a stable secondary shape, and then being controllably actuated to recover their primary shape. Both metal alloys and polymeric materials can have shape memory. In the case of metals, the shape-memory effect arises from thermally induced solid phase transformations in which the lattice structure of the atoms changes, resulting in macroscopic changes in modulus and dimensions. In the case of polymeric materials, the primary shape is obtained after processing and fixed by physical structures or chemical crosslinking. The secondary shape is obtained by deforming the material while in an elastomeric state and that shape is fixed in one of several ways including cooling the polymer below a crystalline, liquid crystalline, or glass transition temperature; by inducing additional covalent or ionic crosslinking, etc. While in the secondary shape some or all of the polymer chains are perturbed from their equilibrium random walk conformation, having a certain degree of bulk orientation. The oriented chains have a certain potential energy, due to their decreased entropy, which provides the driving force for the shape recovery. However, they do not spontaneously recover due to either kinetic effects (if below their lower Tg) or physical restraints (physical or chemical crosslinks). Actuation then occurs for the recovery to the primary shape by removing that restraint, e.g., heating the polymer above its glass transition or melting temperature, removing ionic or covalent crosslinks, etc.

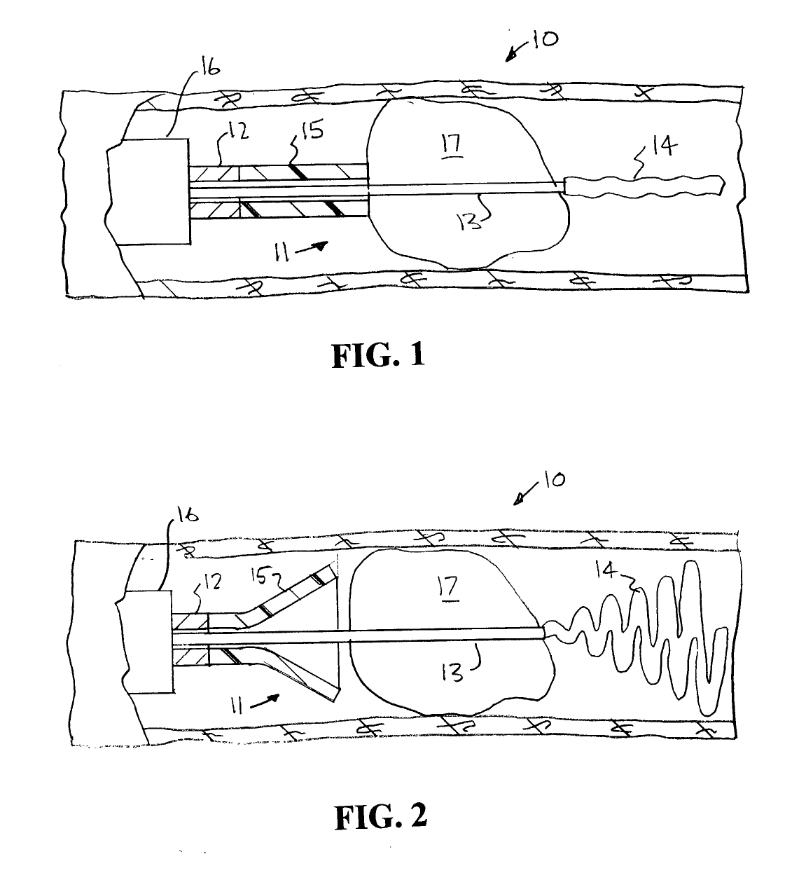

Referring now to FIGS. 1 and 2, a system is illustrated that provides an expandable device used in a the removal of vascular and non-vascular occlusions. The system is generally designated by the reference numeral 10. The system 10 includes an expandable device, designated generally by the reference numeral 11, situated at the distal end of a catheter 12, which can be used to help grip objects when used in a complete system for the removal of vascular and non-vascular occlusions. The expandable device 11 utilizes a shape memory polymer (SMP) actuator, i.e., a polymer which can be formed into a primary or equilibrium shape, re-formed into a stable secondary or stressed shape, and actuated by various means to recover its primary shape.

The SMP actuator may be made of any polymeric material which exhibits a shape-memory effect and is suitable for use in this application. This includes: Thermoplastic SMPs—thermoplastic polymers are those which can be heated into a melt state in which all prior solid shape memory has been lost, processed into a shape, and solidified. If needed be they can be reheated to their melt state and re-processed a number of times. In thermoplastic SMPs, the shape memory effect generally relates to the material having a multiphase structure in which the different phases have different thermal transitions, which may be due to glass transitions, crystalline melting points, liquid crystal-solid transitions, ionomeric transitions, etc. The primary shape is obtained by processing in the melt state above the highest transition temperature and then cooling to a temperature in which either a hard phase or other physical crosslink is formed to lock in that shape. The secondary shape is obtained by bringing the material to a temperature above its actuation temperature but below its melting temperature, mechanically shaping the material into its secondary shape, then cooling it below its actuation temperature while held in its secondary shape. Suitable thermoplastic SMPs include block copolymers (linear di, tri, and multiblocks; alternating; graft), immiscible polymer blends (neat and with coupling agents such as di- or tri-block copolymers), semi-crystalline polymers, and linear polymers with ionomeric groups along the chain or grafted to the chain.

Thermosetting polymers are those which are processed into a part and simultaneously chemically crosslinked, so that the part is essentially one macromolecule. They cannot be re-processed by melting. In thermosetting SMPs the primary shape is obtained during the initial processing step involving crosslinking. The secondary shape is obtained by mechanically reshaping the material at a temperature or condition in which the material is in an elastomeric state. This second shape is locked in by cooling the material below the actuation temperature, which relates to a transition as described above. Suitable thermosetting SMPs include all of the types of materials described under thermoplastic SMPs but which can also be chemically crosslinked to form the primary shape. In addition, crosslinked homopolymers can also be used as SMPs with the actuation temperature typically being the glass transition temperature of the material.

Some polymer undergo a coil to rod transition in their chain conformation by relatively small changes in their environment, such as solvent changes or changes in the ionic character in an aqueous environment. Thermosetting polymers which also undergo rod-coil transitions can be use for this application. Some specific examples of materials include: Polyurethanes as described in U.S. Pat. No. 5,049,591; poly(etheresters); crosslinked acrylic copolymers with monomers consisting of MMA, EA, BA, EMA, BMA, HEMA, AA, and MAA; ethylene-vinyl acetate copolymers.

As shown in FIGS. 1 and 2, the system 10 includes expandable device 11, inner catheter 12, central wire/tube 13, expandable coil 14, SMP expandable catheter end 15, and guiding catheter 16. The system 10 operates through the following system components: a SMP actuator at the end of an inner catheter and an actuation mechanism for heating the SMP above its shape-memory transition temperature, thereby causing shape recovery. Various geometries can be used for the SMP actuator. The actuator will generally have a proximal and a distal end. The proximal end couples to the inner catheter 12, which is generally an elastomeric tube and may have imbedded in it or adhered to it fiber optic cables or wires used in actuation. The coupling of the SMP acuator to the inner catheter 12 may be by pressure fit or mechanical interlock and include the use of adhesives. The SMP actuator will generally have a largely cylindrical geometry in its collapsed or secondary shape, with an inner cylindrical channel suitable for passage of a guide wire device. The distal region of the SMP actuator will have an expanded (primary) shape that is a solid cone, clover leaf or spoke (with spokes straight or helically shaped), or mesh shape. Variations on the expanded shape of the actuator include: the range from an acute angle (0 to 90 degrees) and linear conical expansion to a gradual curved conical expansion; variable number of spokes, ranging from 2 to 32. Variable pitch for helically oriented spokes, ranging from 0 turns (straight) to 90-degree angle with respect to tube axis. Variable number of strands and strand pitch in mesh design.

The SMP actuator can be expanded by several mechanisms for actuation based on movement through a thermal transition including optical heating, convective heating using a heat transfer medium, electrical resistance heating, and inductive heating (see U.S. Pat. Nos. 5,911,737 and 6,059,815). For optical heating optic fibers are used to transport light energy to the SMP actuator. The number and location of the fiber ends are engineered to provide for uniform heating of the SMP. For example, one optic fiber per leaf for a clover leaf geometry is envisioned for the system 10. The light can be absorbed by a suitable dye, which is incorporated into the SMP actuator uniformly, or in a gradient engineered to provide for even heating throughout the actuator geometry. Optical heating may also be accomplished by placing the light absorbing die in an elastomeric coating on the surfaces of the SMP actuator or in an aqueous media which is in contact with the SMP device. Optical heating is preferable from the standpoint that most of the energy applied is used to heat the SMP, minimizing thermal damage to the blood or tissue.

Convective heating may also be used for actuation of the SMP end by pumping a heated saline solution through the interstitial space between the inside of the guide catheter and the outside of the second catheter with the SMP actuator end. This approach can be combined with fiber-optic heating to boost the temperature of the saline to a needed level should cooling during transport through the catheter be a problem.

Inductive heating could be accomplished by filling the SMP with nanoparticules of a material which can selectively absorb RF radiation, converting it to heat. By having an even distribution of particles in the SMP actuator and applying a uniform field the SMP can be quickly and evenly heated for actuation.

The expanded SMP inner catheter end is then used in conjunction with the expanded distal end of the central catheter or guide wire to dislodge the occlusion from the wall of the vascular lumen and hold it so that it can be withdrawn in a substantially whole piece. The inner catheter and central catheter or guide wire may be two separate devices or integrated into a single inner catheter device, which may be especially suitable for smaller dimensions.

Uses for the system 10 include, but are not restricted to, the removal of obstructions from vascular or non-vascular passageways in the body. Such occlusions may include a thrombus (clot), plaque, fatty deposits, and other natural materials as well as fragments of man made devices.

One example of the use of the system 10 involves interventional treatment for acute schemic stroke resulting from a thrombus or embolus lodging in a neuro-vascular lumen. The appliance is inserted into the neuro-vasculature through the carotid artery and brought to the point of occlusion via a guiding or delivery catheter. The coil device at the guide wire end and the inner catheter with SMP tip extend out of the guide catheter to the occlusion. The guide wire with SMP coil tip is then pushed through or past the occlusion. Should it be necessary, both the guide wire with SMP tip and inner catheter with SMP tip can be pushed through the occlusion; however, then the inner catheter would be retracted back to the proximal side of the occlusion. The distal expandable end (coil) of the central tube or guide wire is then actuated to produce a geometry that can be used to pull back the occlusion. The distal end of the inner catheter is then actuated into its expanded shape. The central tube is then pulled back with the clot and the clot is gripped between the expanded end of the central tube/wire and the expanded end of the inner catheter. The clot may be then withdrawn with the device through the guide catheter or if too large, the clot is withdrawn by withdrawing the device and guide catheter.

The potential benefits of this device relate to its potential for improving the outcome for stroke patients. The occurrence of stroke in the US is approaching 700,000/per year, the majority of which are ischemic in nature. Approximately 30% of stroke victims die and ca. 70% of the survivors have some permanent impairment. While this personal cost to victims in terms of quality of life is incalculable, 1994 estimates of direct costs for treatment and lost income were $30 billion (500,000 strokes). Currently, the only FDA approved therapy for acute ischemic stroke involves intraarterial delivery of recombinant tissue plasminogen activator (r-TPA), which acts to prevent and reverse clot formation. This treatment potentially improves the outcome in approximately 30% of cases. However, r-TPA can only be used within the first 3 hours after the occurrence of the stroke and in situations where facilities for handling bleeding complications are present. At the same time the device of this invention may be used within the 3 to 24 hour time frame following the stroke in which partial to complete recovery is possible. Therefore, it could be of benefit for those cases in which r-TPA treatment is not viable or effective, which numbers approximately half of stroke victims.

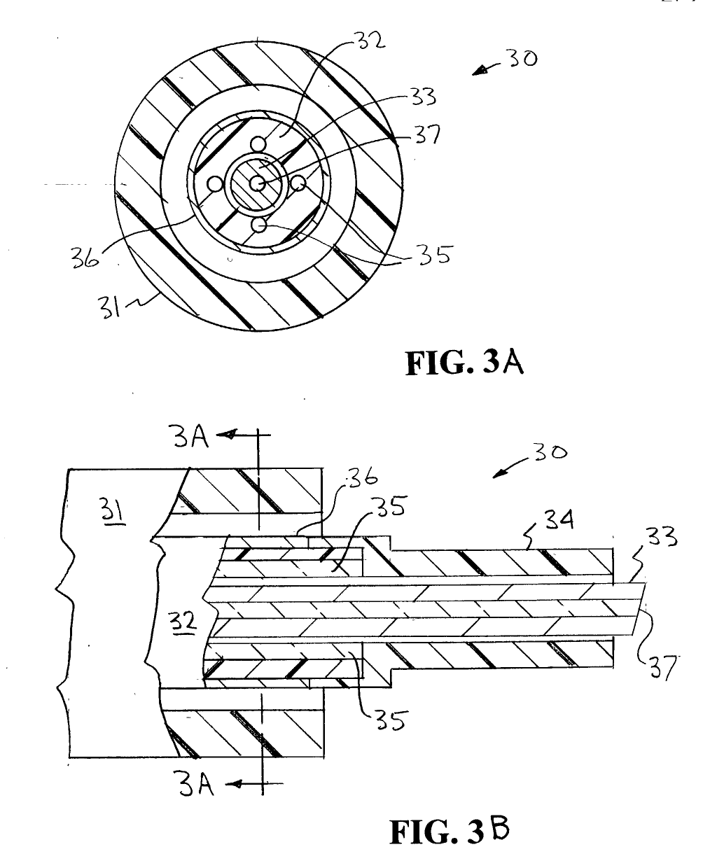

Referring now to FIGS. 3A and 3B, a system that provides another embodiment of an expandable device situated at the distal end of a catheter used in a system for the removal of vascular and non-vascular occlusions is illustrated. The expandable device is constructed of a shape memory polymer (SMP). A SMP is a polymer which can be formed into a primary or equilibrium shape, re-formed into a stable secondary or stressed shape, and actuated by various means to recover its primary shape. The system shown in FIGS. 3A and 3B is generally designated by the reference numeral 30. The system 30 operates through the following system components: a SMP actuator at the end of an inner catheter 32 and an actuation mechanism for heating the SMP above its shape-memory transition temperature, thereby causing shape recovery. Various geometries can be used for the SMP actuator. The actuator will generally have a proximal and a distal end. The proximal end couples to an inner catheter, which is generally an elastomeric tube and may have imbedded in it or adhered to it fiber optic cables or wires used in actuation. The coupling of the SMP acuator to the inner catheter may be by pressure fit or mechanical interlock and include the use of adhesives. The SMP actuator will generally have a largely cylindrical geometry in its collapsed or secondary shape, with an inner cylindrical channel suitable for passage of a guide wire device. The distal region 34 of the SMP actuator will have an expanded (primary) shape that is a solid cone, clover leaf or spoke (with spokes straight or helically shaped), or mesh shape. Variations on the expanded shape of the actuator include: the range from an acute angle (0 to 90 degrees) and linear conical expansion to a gradual curved conical expansion; variable number of spokes, ranging from 2 to 64. Variable pitch for helically oriented spokes, ranging from 0 turns (straight) to 90-degree angle with respect to tube axis. Variable number of strands and strand pitch in mesh design.

The SMP actuator can be expanded by several mechanisms for actuation based on movement through a thermal transition including optical heating, convective heating using a heat transfer medium, electrical resistance heating, and inductive heating. The heat transfer mechanism shown in the system 30 includes at least one optical fiber 35. Optical heating may also be accomplished by placing the light absorbing die in an elastomeric coating on the surfaces of the SMP actuator or in an aqueous media which is in contact with the SMP device. Optical heating is preferable from the standpoint that most of the energy applied is used to heat the SMP, minimizing thermal damage to the blood or tissue. The heat transfer mechanism can be other heat transfer mechanisms including induced heating, electrical induction heating, electrothermal heating, utilizing an acoustic energy source, utilizing ultrasonic energy, utilizing radio frequency (RF) energy, and utilizing magnetic energy.

The system 30 includes the following components, outer catheter 31, inner catheter 32, guide wire 33, SMP expandable catheter end 34, optic fibers 35, shield 36 and optic fiber 37. FIG. 3A is an end view of the system 30 and FIG. 3B is a side view. The system 30 is used to remove vascular and non-vascular occlusions. The system 30 operates according to a number of steps. The first step is passing a transport vehicle such as guide wire 33, inner catheter 32, outer catheter 31 and the shape memory polymer material through the conduit. The next step is passing the shape memory polymer material through or around the matter. This is accomplished as illustrated in FIG. 1 where the central wire/tube and expandable coil are passed into the occlusion until the expandable coil is in a position to engage the occlusion when the coil is expanded. The nest step is transmitting energy to the shape memory polymer material for moving the shape memory polymer material from the first unexpanded shape to a second and different shape wherein it forms an expanded coil. The next step is withdrawing the transport vehicle and the shape memory polymer material through the conduit carrying matter that forms the occlusion.

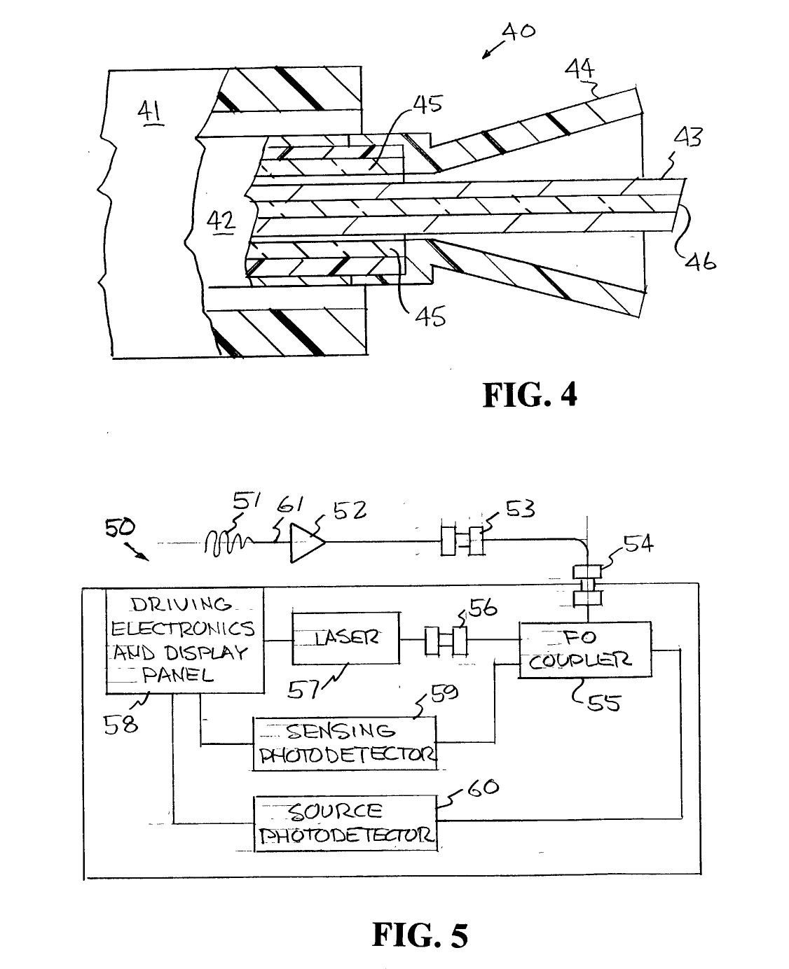

Referring now to FIG. 4, a system the illustrates additional details of an expandable device situated at the distal end of a catheter used in a system for the removal of vascular and non-vascular occlusions is illustrated. The expandable device is generally designated by the reference numeral 40 and is constructed of a shape memory polymer (SMP). A SMP is a polymer which can be formed into a primary or equilibrium shape, re-formed into a stable secondary or stressed shape, and actuated by various means to recover its primary shape. The expandable device 40 includes an inner catheter 42 and a central wire/tube 43. An expandable coil is connected to the distal end of inner catheter 42, central wire/tube 43. A SMP expandable catheter end 44 is connected to outer catheter 41.

In operation the catheters 41 and 42 with a shape memory material is transported to the site of the matter to be removed. The shape memory material is passed through or around the matter to be removed. For example, the matter to be removed could be from a vessel with a blockage. The blockage could be a blood clot, plaque, other emboli, or other blockage. The support structure including catheters 41 and 42 and guide wire 43 with a shape memory material actuator on its distal end is inserted through or around the blockage. The shape memory material actuator is used to remove the blockage from the vascular system. Heat is utilized to activate the shape memory material and expand the shape memory material on both the guide wire and inner catheter. By withdrawing the catheter and the shape memory material through the vessel the matter is carried from the vessel.

Actuation is achieved by heating the shape memory material. The shape memory material can be heated using various systems. These systems include induced resistive heating by an external wave field, such as by an associated magnetic or radio frequency (RF) source, external heating through electrical induction or electrothermal heating, with local or remote ultrasonics or other acoustic means of energy transfer, or by converting optical energy into thermal energy that allows the stored energy in the shape memory material to be released. The heating of the SMP can be accomplished by an operatively connected or embedded mechanism which is powered by the absorption of applied energy in the form of light, electric fields, magnetic fields, RF fields, EM waves, acoustic/ultrasound waves, electric current flow (DC: resistive heating, AC: inductive or dielectric heating), chemical reactions and/or other heating mechanisms such as nuclear heating, etc. The optical energy is absorbed by the shape memory material and converted into thermal energy that heats the shape memory material above its transition temperature and the shape memory material moves to its primary shape, resulting in opto-mechanical actuation.

When the shape memory material actuator is drawn backward it results in the removal of the blockage from the vessel. The catheter with the shape memory material was transported to the site of the matter to be removed. The shape memory material was passed through or around the matter. Heat was utilized to activate the shape memory material and expand the shape memory material coil. The SMP expandable catheter end 44 contacts and assists in removal of the matter. By withdrawing the support structure and the shape memory material through the vessel the matter was carried from the vessel.

Referring now to FIG. 5, a schematic overview of an ischemic stroke treatment system 50 is show. Laser light from laser 57 is transmitted through a multimode optical fiber 61, a fiber coupler, an extension fiber that enters the sterile surgical field, and a fiber pusher with the SMP actuators at the distal tip of the central tube and inner catheter. Separate fibers are used to actuate the central tube tip and the inner catheter tip for independent control. A small amount of laser light is reflected form the fiber-coil interface back through the coupler into the photodetector 59. Source fluctuations may be monitored by the source photodetector 60. As the laser light heats the SMP in the distal tip of the catheter, the expandable coil deploys. Detection of the actuation can be fed back to the operator (or computer). The SMP movement causes the reflected signal to decrease. The changes in the reflected signal can be used to control the driving current of the laser or to alert an operator of the status of the actuator (e.g., open or closed).

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A shape memory polymer device for acting upon a material in a conduit, comprising:
a transport vehicle adapted to move through said conduit,
a shape memory material operatively connected to said transport vehicle, said shape memory material adapted to move through or around said material while in a first shape and capable to transition from a first shape to a second and different shape for acting upon said material, and
a heat transfer mechanism operatively connected to said shape memory material, adapted to transfer heat to said shape memory material to transition said shape memory material from said first shape to said second shape.

2. The shape memory device of claim 1, wherein said conduit is a vascular or a non-vascular conduit.

3. The shape memory device of claim 2, wherein said conduit is a vascular conduit.

4. The shape memory device of claim 3, wherein said shape memory material is in the general shape of a wire.

5. The shape memory device of claim 3, wherein said shape memory material in the general shape a wire that can be expanded to form an expanded coil.

6. The shape memory device of claim 3, wherein said first shape is an unexpanded coil shape and said second shape is an expanded coil shape.

7. The shape memory device of claim 3, including a second shape memory material operatively connected to said transport vehicle adapted to move through said conduit and is capable to transition from a first shape to a second and different shape for acting upon said material.

8. The shape memory device of claim 7, wherein said second shape memory material is adapted to move through said conduit and capable to transition from a first shape to a second and different shape so that the material is gripped between the two shape memory devices.

9. The shape memory device of claim 3, wherein said heat transfer mechanism includes an optical fiber.

10. The shape memory device of claim 3, wherein said heat transfer mechanism is induced heating.

11. The shape memory device of claim 3, wherein said heat transfer mechanism is electrical induction heating.

12. The shape memory device of claim 3, wherein said heat transfer mechanism is electrothermal heating.

13. The shape memory device of claim 3, wherein said heat transfer mechanism utilizes acoustic energy source.

14. The shape memory device of claim 3, wherein said heat transfer mechanism utilizes ultrasonic energy.

15. The shape memory device of claim 3, wherein said heat transfer mechanism utilizes radio frequency (RF) energy.

16. The shape memory device of claim 3, wherein said heat transfer mechanism utilizes magnetic energy.

17. The shape memory device of claim 9, including a catheter operatively connected to said optical fiber and said shape memory material.

18. The shape memory device of claim 9, including a delivery catheter operatively connected to said transport vehicle, to said optical fiber, and to said shape memory material.

19. A shape memory polymer device for acting upon a material in a conduit, comprising:
a transport vehicle adapted to move through said conduit,
a first shape memory material operatively connected to said transport vehicle, said first shape memory material adapted to move through or around said material while in a first shape and capable of transitioning from said first shape to a second and different shape for acting upon said material,
a second shape memory material operatively connected to said transport vehicle adapted to move through said conduit and capable of transitioning from an initial shape to a different shape for acting upon said material, and
a heat transfer mechanism operatively connected to said first shape memory material and said second shape memory material, adapted to transfer heat to said first shape memory material and said second shape memory material to transition said first shape memory material from said first shape to said second shape and to transition said second shape memory material from said initial shape to said different shape.

20. The shape memory device of claim 19, wherein said conduit is a vascular or a non-vascular conduit.

21. The shape memory device of claim 2, wherein said conduit is a vascular conduit.

22. The shape memory device of claim 3, wherein said material is gripped between said first shape memory material and said second shape memory material.

23. A method of removing matter from a conduit, comprising the steps of:
passing a transport vehicle and a shape memory polymer material through said conduit,
passing said shape memory polymer material through or around said matter,
transmitting energy to said shape memory polymer material for moving said shape memory polymer material from a first shape to a second and different shape, and
withdrawing said transport vehicle and said shape memory polymer material through said conduit carrying said matter.

24. The method of removing matter from a conduit of claim 23 wherein said conduit is a vascular or a non-vascular conduit.

25. The method of removing matter from a conduit of claim 24 wherein said conduit is a vascular conduit.

26. The method of removing matter from a conduit of claim 23 wherein said first shape is an unexpanded coil shape and said second shape is an expanded coil shape.

27. The method of removing matter from a conduit of claim 23 wherein said step of transmitting energy to said shape memory polymer material includes transmitting light through an optical fiber.

28. The method of removing matter from a conduit of claim 23 wherein an induced heating mechanism is used in said step of transmitting energy to said shape memory polymer material.

29. The method of removing matter from a conduit of claim 23 wherein an electrical induction heating mechanism is used in said step of transmitting energy to said shape memory polymer material.

30. The method of removing matter from a conduit of claim 23 wherein an electrothermal heating mechanism is used in said step of transmitting energy to said shape memory polymer material.

31. The method of removing matter from a conduit of claim 23 wherein an acoustic energy transfer source is used in said step of transmitting energy to said shape memory polymer material.

32. The method of removing matter from a conduit of claim 23 wherein an ultrasonic energy transfer source is used in said step of transmitting energy to said shape memory polymer material.

33. The method of removing matter from a conduit of claim 23 wherein a radio frequency (RF) energy transfer source is used in said step of transmitting energy to said shape memory polymer material.

34. The method of removing matter from a conduit of claim 23 wherein a magnetic energy transfer source is used in said step of transmitting energy to said shape memory polymer material.

35. The method of removing matter from a conduit of claim 23 including the step of using a delivery catheter to carry said catheter and said shape memory polymer material to said matter.

36. The method of removing matter from a conduit of claim 23 including the steps of transmitting light energy through an optical fiber to said shape memory polymer material and using a catheter to direct said shape memory polymer material to said matter.

37. The method of removing matter from a conduit of claim 36 including the step of using a guide catheter to move said catheter, said optical fiber, and said shape memory material through said conduit.

38. The method of removing matter from a conduit of claim 23 including the step of using a second shape memory polymer material to grip said matter.

39. A method of removing matter from a conduit, comprising the steps of:
passing a transport vehicle carrying a first shape memory polymer material and a second shape memory material through said conduit,
passing said first shape memory polymer material through or around said matter,
positioning said second shape memory polymer material adjacent said matter,
transmitting energy to said first shape memory polymer material for moving said first shape memory polymer material from a first shape to a second and different shape and for moving said second shape memory material from an initial shape to a different shape, and
withdrawing said transport vehicle, said first shape memory polymer material, and said second shape memory polymer material through said conduit carrying said matter.

40. The method of removing matter from a conduit of claim 39 wherein said first shape memory polymer material and said second shape memory polymer material grip said matter.
